(19) United States
(12) Patent Application Publication
(10) Pub. No.: US 2007/0218052 A1
(43) Pub. Date: Sep. 20, 2007

(54) Novel lgG3 Antibodies for Stimulating Phagocytosis

(75) Inventors: Christophe De Romeuf, Lille (FR); Sylvie Jorieux, Villeneuve-D'Ascq (FR); Dominique Bourel, La Madeleine (FR); Philippe Klein, Lille (FR); Nicolas Bihoreau, Orsay (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Labopratoire Francais du Fractionnement et des Biotechnologies, Les Ulis (FR)

(21) Appl. No.: 10/575,218

(22) PCT Filed: Oct. 18, 2004
(86) PCT No.: PCT/FR04/02657

§ 371 Date: Feb. 20, 2007

(30) Foreign Application Priority Data
Oct. 16, 2003 (FR) 0312087

(51) Int. Cl. A61K 39/395; C07K 16/00

(52) U.S. Cl. 424/133.1; 424/141.1; 530/387.1

(57) ABSTRACT

The invention relates to human or humanised, chimeric, monoclonal, class IgG3 antibodies produced in a cell line of rat myeloma, especially line YB2/0. Said antibodies have a strong phagocytosis activity and can be administered for the treatment of cancers and infections.

This invention relates to the use of chimeric, humanised or human class IgG3 monoclonal antibodies produced in a cell line of rat myeloma, particularly YB2/0 (ATCC No. CRL 1662) or a derived or modified line of YB2/0 for preparation of a medicine for the treatment of different cancer and infectious pathologies. These antibodies have a strong phagocytosis activity and can be administered to treat cancers and infections.

At the moment, the large majority of therapeutic monoclonal antibodies that have been marketed or on which clinical tests are being carried out belong to the IgG1 class. However, other antibody subclasses apart from IgG1 could also have advantages for the treatment of some pathologies.

IgG3s in particular have particular effector capabilities and certainly play an important role in vivo. Although they only represent 7% of IgGs in human plasma, their proportion is increased during some immune responses, for example following some viral infections (Basic and clinical aspects of IgG subclasses. Volume editor, F. Shakib, Basel; New York: Karger, 1986 (Monographs in Allergy; Vol 19, Pages 122-133), parasite infections (J Infect Dis. 2003 Mar. 1; 187(5): 862-5, 2003, Immunoglobin G (IgG) responses to Plasmodium falciparum glycosylphosphatidylinositols are short-lived and predominantly of the IgG3 subclass. Boutlis C S, Fagan P K, Gowda D C, Lagog M, Mgone C S, Bockarie M J, Anstey N M) or following immunisations against the Rh(D) antigen (Iyer Y S, Kulkarni S V, Gupte S C. Distribution of IgG subtypes in maternal anti-D sera and their prognostic value in Rh haemolytic disease of the new-born. Acta Haematol. 1992; 88(2-3); 78-81).

The therapeutic use of IgG3s has been very limited up to now. They are used particularly in the preventive treatment of haemolytic disease of the new-born, since firstly polyclonal anti-D antibodies used at the moment are composed of about 20 to 30% of IgG3 and secondly, clinical studies using an IgG3 anti-D monoclonal antibody have already been carried out with encouraging results in terms of clearance of positive Rh red cells (Clin Exp Immunol. 2003 April; 132(1): 81-6. Clearance of red cells by monoclonal IgG3 anti-D in vivo is affected by the VF polymorphism of Fc gamma RIIIa (CD16), Kumpel B M, De Haas M, Koene H R, Van De Winkel J G, Goodrick M J).

Although the action mechanism of anti-D polyclonal antibodies leading to the lack of immunisation of the mother is not known, many studies have attempted to demonstrate the corresponding roles of anti-D IgG1 and IgG3. For example, it has been demonstrated that formation of rosettes between effector cells such as monocytes, T CD8 lymphocytes, B lymphocytes and NK cells with Rhesus positive D red cells, was faster and more important with anti-D IgG3s than with IgG1s. These differences can be explained by the longer hinge region of IgG3s than IgG1s. This structure would facilitate the formation of bridges between negatively charged red cells and effector cells (Vox. Sang. 1989; 56(2): 101-3, Rate of interaction of IgG1 and IgG3 sensitised red cells with monocytes in the phagocytosis assay, Brojer E, Merry A H, Zupanska B; Immunology; 1992 July; 76(3): 446-51. The functional. activity of Fc gamma RII and Fc gamma RIII on subsets of human lymphocytes, Hadley A G, Zupanska B, Kumpel B M, Leader K A).

The existence of competition between IgG1s and IgG3s thus suggesting that these two IgG subclasses could recognise and activate the same receptor Fc was mentioned in other studies (Immunology, 1989 April; 66(4): 491-8, Distinctive role of IgG1 and IgG3 isotypes in Fc gamma R-mediated functions, Rozsnyay Z, Sarmay G, Walker M, Maslanka K, Valasek Z, Jefferis R, Gergely J).

During parasite infections such as Plasmodium falciparum and during bacterial infections, an IgG3 type response is observed and is associated with production of IgG1 against proteic antigens. Similarly, in the case of the anti-polysaccharidic response with bacterial origin (anti-LPS), even if the predominant sub-class consists of IgG2s, there is a strong IgG1 type response and a more limited IgG3 response.

For cancer pathologies, there are no data on production of IgG3s in patients, although anti-tumour treatments using IgG3s coupled or not coupled to cytokines were used experimentally (Kemminer S E, Conradt H S, Nimtz M, Sagi D, Peter-Katalinic J, Diekmann O, Drmic I, Muthing J Biotechnol Prog. 2001 September-October; 17(5): 809-21. Production and molecular characterisation of clinical phase I anti-melanoma mouse IgG3 monoclonal antibody R24). (Peng L S, Penichet M L, Dela Cruz J S, Sampogna S L, Morrison S L. J Interferon Cytokine Res. 2001 September; 21(9): 709-20. mechanism of anti-tumour activity of a single-chain interleukin-12 IgG3 antibody fusion protein (mscIL-12.her2.IgG3)).

Line YB2/0 was selected for several years for its ability to confer improved functional properties on IgG1s produced. We have demonstrated the importance of selecting cell lines capable of producing antibodies with a strong ADCC activity through the FcγRIII (CD16) receptor, in our application WO 01/77181. We also found that a modification to the glycosylation of the constant part of antibodies produced in rat myeloma lines such as YB2/0 further improved the ADCC activity.

Glycannic structures of the said antibodies are of biantenna type, characterised by short chains, weak sialylation and weak fucosylation.

We also discovered that the fact that there is a strong interaction with CD16 has the advantage that it also induces the production of cytokines, particularly the production of IFNγ and/or other cytokines or chemokines.

The two characteristics mentioned above are complementary. The production of IFNγ or other cytokines and/or chemokines by effector cells induced by the selected antibodies can reinforce the therapeutic effect by stimulating effector mechanisms of the immunity system other than the ADCC in treated patients. The action mechanism for such a stimulation is probably due to positive autocrine regulation of effector cells. It could be postulated that the antibodies bonding to CD16 induce a cytotoxic activity and the production of IFNγ or other cytokines/chemokines that eventually increase the cytotoxic activity even further.

Within the scope of this invention, an anti-D IgG3 was expressed in a rat myeloma line in order to determine if this line, particularly YB2/0, can confer improved functional properties on the antibodies produced, as is the case for IgG1s.

Our results indicate that the IgG3s thus produced have a capability for bonding to CD16 comparable to the capability of IgG1s. Nevertheless, this increase in bonding to CD16 is not correlated to a release of cytokines and induces a weaker potentialisation of ADCC than is observed with IgG1s. However, in some “in vitro” conditions, in other words in the presence of fixed red cells and only with a high concentration of antibodies, IgG3s produced in YB2/0 appear capable of inducing release of cytokines. On the other hand, we quite unexpectedly observed that phagocytosis is increased.

### DESCRIPTION

Thus, according to a first aspect, the invention relates to chimeric, humanised or human class IgG3 monoclonal antibodies characterised in that they are produced in a cell line of rat myeloma. Preferably, the said IgG3s are produced in line YB2/0 (ATCC No. CRL 1662) or a derived or modified line of YB2/0.

In such antibodies, the glycannic structure of the Fc region corresponds to a biantenna type, with short chains, weak sialylation and weak fucosylation.

Furthermore, the content of intermediate GlcNac is not zero.

Such antibodies are selected particularly from among the following forms:

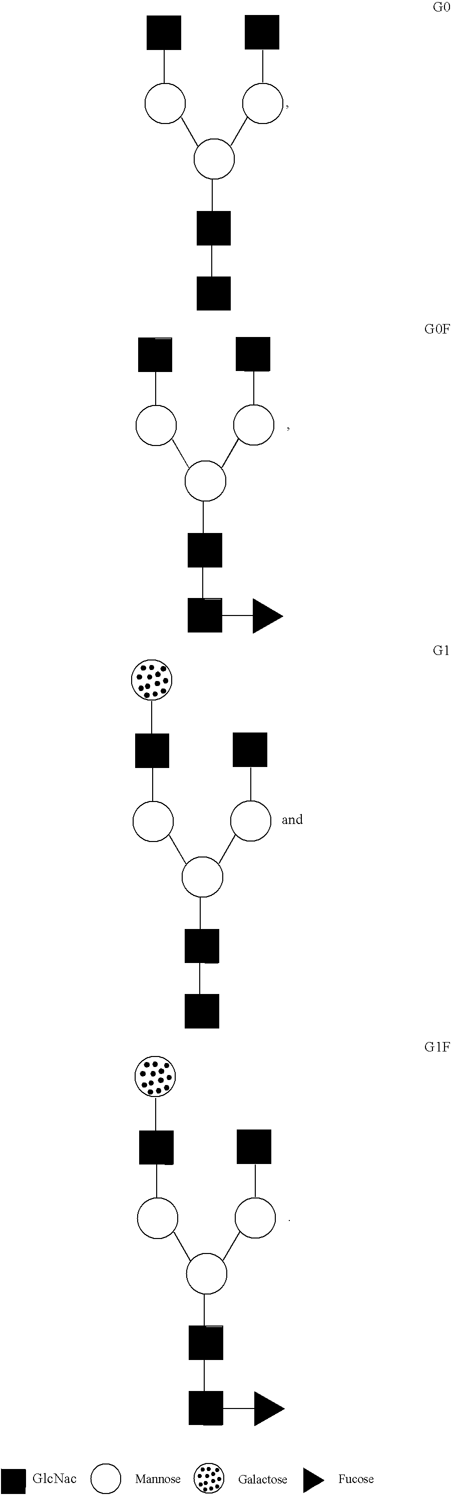

Thus, the invention relates to class IgG3 monoclonal antibodies in which the fucose content is less than 65%, 60%, 50%, 40% or 35%. Preferably, the fucose content is between 20% and 45%, or better between 25% and 40%. For example, the fucose content is less than 35%.

The invention also relates to class IgG3 antibodies with the glycosylation profile mentioned above produced in equivalent biological systems, particularly in genetically modified or transformed plant or non-human animal cells, for example by the introduction of a sequence expressing one or several glycosyl transferases so as to obtain antibodies with a profile essentially similar to the profile of glycosylation obtained in YB2/0.

IgG3s produced in the YB2/0 line have particular functional characteristics, that do not occur in lines such as CHO for example:

a) strong bonding to CD16 which is comparable to bonding of IgG1s produced in the same cell line,

b) a capability of inducing an inhibition of the release of cytokines induced by IgG1s.

c) a greater capability of IgG3s produced in YB2/0 to induce release of gamma IFN, IL6 and alpha TNF than IgG3s produced in CHO; and a lower capability of YB2/0 IgG3s to induce release of alpha TNF and gamma IFN than YB2/0 IgG1s.

d) a potentialisation of phagocytosis.

The class IgG3 antibody according to the invention may be selected as an example from among antibodies directed against CD2, CD3, CD4, CD5, CD7, CD8, CD11, CD18, CD19, CD20, CD25, CD45 and CD52 such as Campath-1H®, CD30, CD33, CD38 or CD44.

Other antibodies can be selected from among anti Ep-CAM, anti HER2, anti HER1, anti GD3, anti CA125, anti GD, anti GD2, anti CD-23 and anti Protein C; anti KIR3□L2, anti-EGFR, anti-idiotypes specific for inhibitors for example for coagulation factors, HIV, HBV, HCV and RSV antivirals.

A second aspect of the invention relates to a process for the production of chimeric, humanised or human class IgG3 monoclonal antibodies with the functional characteristics mentioned above comprising transfection of a cell line preferably of rat myeloma, line YB2/0 (ATCC No. CRL 1662) or a derived or modified line of YB2/0 with one or several vectors comprising coding sequences for heavy and lightweight chains of class IgG3 antibodies, the expression of the said antibodies in the transfected cell line, extraction and purification of the antibodies.

Preferably, a system with two expression vectors (for example vectors derived from RSV) are used, one coding vector for heavy chains and the other coding vector for light chains. Advantageously, a different selection marker is present in each vector. Specific constructions are shown in FIG. 1. The invention also relates to the system described above in which the heavy and light chains are produced in equimolar quantity.

The construction of expression vectors may be used according to procedures known to those skilled in the art (Molecular Cloning: A Laboratory Manual, Second Edition, Maniatis et al, Cold Spring Harbor).

The two vectors in the rat myeloma line can be co-transfected using an equimolar quantity and using standard procedures such as precipitation with calcium phosphate or lipofectine. The transfected lines are then selected in appropriate culture media.

Obviously, other strategies can be used, and particularly the use of a single coding vector for all chains in the antibody.

In a third aspect, the invention relates to cell lines of rat myeloma, and particularly YB2/0 and derived lines transfected by one or several vector(s) enabling the expression of a functional IgG3. The invention also relates to cells that have been transfected by one or several vector(s) described above. These cells are characterised in that they produce IgG3s with the glycosylation profile mentioned above and at least one of the properties a) to d) described above. A cell derived from a line described above is also another purpose of the invention.

According to a fourth aspect, the invention relates to the use of IgG3s described above, particularly IgG3s expressed in YB2/0 for the preparation of a medicine.

More specifically, the invention relates to the use of chimeric, humanised or human class IgG3 monoclonal antibodies produced in a cell line of rat myeloma, particularly YB2/0 (ATCC No. CRL 1662), or a derived or modified line of YB2/0, for preparation of a medicine intended for the treatment of different cancer pathologies or different infectious pathologies with viral, bacterial or infectious parasite infections.

In another aspect of the invention, these antibodies may be used for the preparation of a medicine intended for the prevention of foetal maternal alloimmunisation.

Preferably, the patients concerned are patients with a weak response to treatment with an IgG1 or an IgG3 expressed in CHO.

“Patients with weak responses” means treated patients in a so-called stable condition, with less than 50% reduction and less than 25% increase in lesions, and no new lesions. This group of patients also includes patients for which no response is observed (progress of the disease that could lead to death). For infectious diseases, these patients are patients for whom a conventional treatment reduces the viral or bacterial charge by less than 50%.

Advantageously, the antibody can be used in patients with a late diagnosis.

Cancer pathologies that can be particularly advantageously treated using antibodies according to the invention are chosen from among the group comprising neuroectodermal tumours, colorectal cancers, melanomas, breast cancer, leukaemia and particularly HCL (Hairy Cell Leukaemia), lymphomas such as DLBCL (Primary Diffuse Large B-Cell Lymphomas), acute leukaemia, osteosarcomas, cancer and particularly lung cancer, this list not being exhaustive.

In one particular aspect of the invention, cancer pathologies treated according to the invention are associated with viral or bacterial infections such as cancer of the prostate (Lightfoot N, Conlon M, Kreiger N, Sass-Kortsak A, Purdham J, Darlington G. Medical History, sexual and maturational factors and prostate cancer risk. Ann Epidemiol, 2004 October; 14(9): 655-662; Huycke M M, Gaskins H R. Commensal bacteria, redox stress, and colorectal cancer: mechanisms and models. Exp Biol Med (Marywood), 2004 July; 229(7): 586-97), leukaemias and Kaposi's sarcoma. Infectious agents found in infectious diseases associated with cancer can be Candida, Achromobacter or Alcaligenes (Aisenberg G, Rolston K V, Safdar A. Bacteremia caused by Achromobacter and Alcaligenes species in 46 patients with cancer (1989-2003). Cancer 2004 Sep. 23; Boktour M R, Kontoyiannis D P, Hanna H A, Hachem R Y, Girgawy E, Bodey G P, Raad I I. Multiple-species candidemia in patients with cancer. Cancer, 2004 Aug. 31) or the Epstein-Barr virus.

Infectious pathologies that can advantageously be treated with the antibody according to the invention include diphtheria, viral hemorrhagic fevers, typhoid fever, influenza, hepatitis B and C, respiratory infections due to RSV, infections due to HIV and CMV, legionnaires' disease, Leishmaniasis, leprosy, rabies, AIDS or tuberculosis, this list not being limitative.

IgG3s according to the invention have an advantage for these uses due to their strong bonding to the low affinity receptor Fc (CD16) and/or their capability of inducing a phagocytosis.

In one particular aspect of the invention, the medicine according to the invention will be used in combination with an IgG1. The use of IgG3s according to the invention as described above is particularly advantageous in this aspect of the invention for the capability of these IgG3s to negatively modulate the release of cytokines induced by IgG1, and particularly the contents of gamma IFN, alpha TNF and/or IL6.

Thus in one particular aspect of the invention, IgG3s like those described above are used for the preparation of a medicine for the treatment of cancer pathologies in patients with a “cytokine release syndrome”, particularly in patients treated by an IgG1 produced in YB2/0. This application makes use of the capability of the said IgG3s to negatively modulate the release of cytokines. For example, the appearance of hypothermia, acute renal necrosis and diseases of the liver due to “cytokine release syndrome” induced by the administration of an anti-CD3 monoclonal antibody, for example 145-2C11 (Alegre M L et al, Immunol. 1991 Feb. 15; 146 (4): 1184-91; Chatenoud L. Anti-CD3 antibodies: towards clinical antigen-specific immunomodulation. Curr Opin Pharmacol. 2004 August; 4 (4): 403-7; Yamada-Ohnishi Y, Azuma H, Urushibara N, Yamaguchi M, Fujihara M, Kobata T, Ikeda H. Cytotoxic Difference of T Cells Expanded with Anti-CD3 Monoclonal Antibody in the Presence and Absence of Anti-CD28 Monoclonal Antibody, Stem Cells Dev. 2004 June; 13(3): 315-22).

Alternately, the invention aims at the use of an IgG3 described above that may be an anti-CD20 to prevent the appearance of the “cytokine release syndrome” in patients treated with Rituximab® (IDEC-C2B8); Winkler U et al, Cytokine release syndrome in patients with B-cell chronic lymphocytic leukaemia and high lymphocyte counts after treatment with an anti-CD20 monoclonal antibody, Blood 1999 October; 94 (7): 2217-24.

Alternatively, the IgG3 according to the invention is useful to prevent the undesirable effects of the CAMPATH® or OKT3 antibody.

Administration of CAMPATH 1-H that bonds to the CD52 on lymphocytes and monocytes, induces the release of TNF, IFN, IL-6 leading to the “cytokine release syndrome”, Mark G. Wing et al, Mechanism of First-Dose Cytokine-Release Syndrome by CAMPATH 1-H: Involvement of CD16 (FCRIII) and CD11a/CD18 (LFA-1) on NK cells, J. Clin. Invest, Volume 98, Number 12, December 1996, 2819-2826. Similarly OKT3 that bonds to CD3 is also described as inducing the cytokine release syndrome (First M R, Schroeder T J, Hariharan S. OKT3-induced cytokine release syndrome: renal effects (cytokine nephropathy). Transplant Proc. 1993 April; 25 (Suppl 1): 25-6).

Another purpose of the invention is to provide a process for modulating the release of cytokines induced by an IgG1 by adding IgG3s produced in a cell line of rat myeloma, particularly YB2/0, to the biological system containing the said IgG1s.

The combination of an IgG1 and an IgG3 has an important therapeutic advantage because it can reduce secondary effects due to IgG1 without significantly affecting its cytotoxic capabilities and increase the therapeutic effect through phagocytosis.

In one particular aspect of the invention, IgG1s for which release of cytokines is modulated are produced in a cell line of rat myeloma and particularly in YB2/0.

In a final aspect, the purpose of the invention is a pharmaceutical composition of therapeutic antibodies comprising IgG1s, IgG3s and at least one excipient.

Advantageously, the at least one of these IgGs (IgG1 or IgG3) is produced in a cell line of rat myeloma, and particularly YB2/0.

### LEGENDS AND TITLES OF THE FIGURES

FIG. 1: Diagram showing expression vectors

FIG. 2: Illustration of antibodies produced

FIG. 3: Interaction with Jurkat CD16 cells of antibodies coated on red cells fixed on the microtitration plate.

The x axis represents bonding of antibodies to red cells and the y axis represents bonding to the CD16.

FIG. 4: Bonding of IgG3s to Jurkat CD16 cells in the absence of targets.

FIG. 5: Release of IL-2 induced by IgG1s and IgG3s expressed in YB2/0 after interaction with Jurkat CD16 cells.

FIG. 6: ADCC activity of IgG1 and IgG3 anti-D antibodies in the presence of PBMC and polyvalent immunoglobulins.

FIG. 7: ADCC activity in the presence of NK cells and IgG1 and IgG3 anti-D antibodies.

FIG. 8: Release of IL-2 by Jurkat CD16 cells induced by anti-Rhesus IgG1s and IgG3s (red cells in suspension). Effect of the addition of the different IgG3s on IL21 release induced by YB2/0 IgG1s.

FIG. 9: Release of cytokines induced by antibodies in the presence of NK cells or monocytes.

FIG. 10: Percentage of THP 1 cells that have phagocyted one or several red cells.

### EXAMPLES

### Example 1

### Obtaining Different Class IgG3 Anti-D Antibodies

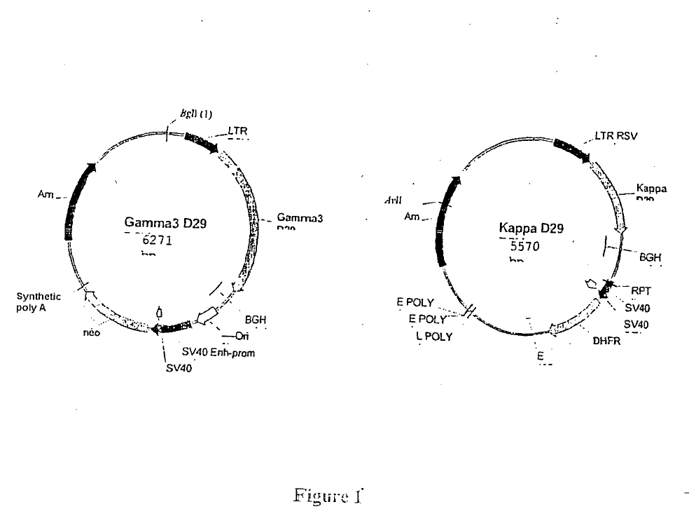

FIG. 1 shows the construction of expression vectors to produce two recombinant antibodies. After the construction of these expression vectors, transformants producing. D29 IgG3s with anti-D specificity were obtained in the YB2/0 and CHO lines.

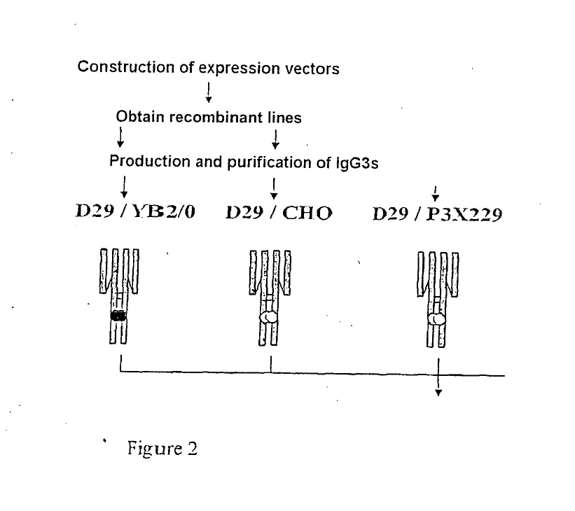

The different antibodies thus produced are shown diagrammatically in FIG. 2:

Antibody 1: D29 IgG3s in the YB2/0, D29-YB2/0 line

Antibody 2: IgG3 expressed in the CHO (reference line for the industrial production of recombinant proteins), D29-CHO line.

Antibody 3: IgG3 (D29 produced by a lymphocyte B merged with P3X229), D29-P3X229.

### Example 2

### Study on Bonding of the Three Antibodies in Example 1 to Jurkat CD16 Cells (CFC)

This test was set up to evaluate the capability of anti-D antibodies to bond onto the CD16 receptor (Fc gamma RIIIa) expressed on Jurkat CD16 cells.

The first step consists of making the anti-D antibody react with Rhesus antigens expressed on the surface of Rhesus positive red cell membranes previously coated on 96 well plates with a round bottom (bonding by Fab). This bonding is detected at the same time by a human anti-IgG antibody marked with alkaline phosphatase.

The second step (after bonding of the antibody to its antigen) consists of adding Jurkat CD16 cells that will be able to interact with the Fc part of the antibodies. After centrifuging, a score (bonding index of 1 to 3) corresponding to the Jurkat CD16 cells that are bonded to the antibodies is estimated visually.

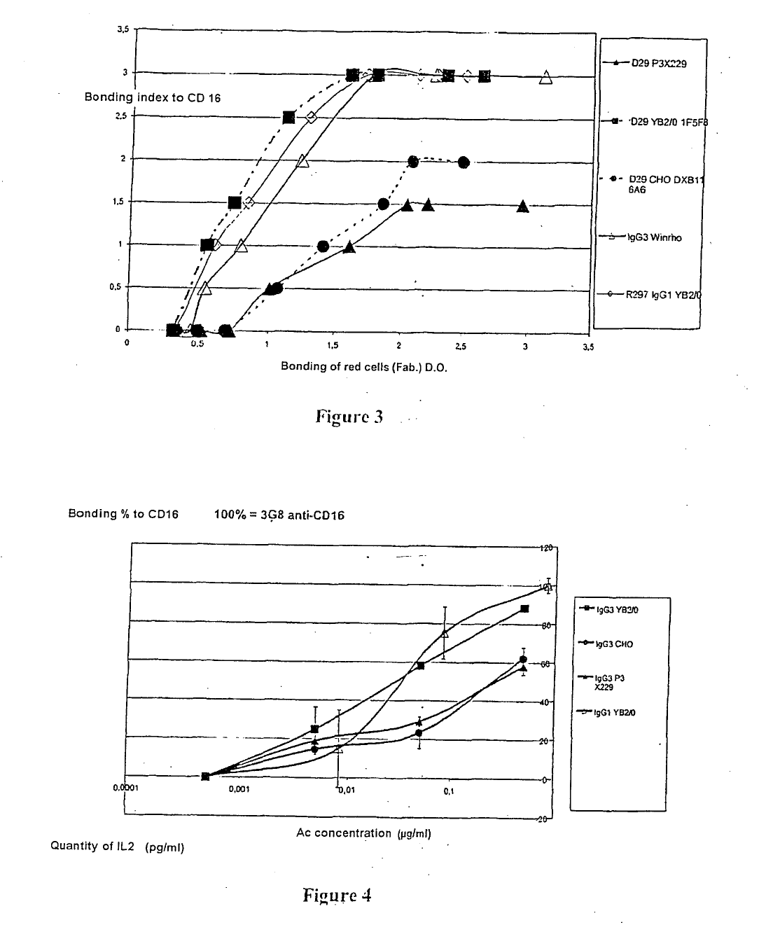

FIG. 3 shows the results.

It can be concluded from the results that the expression of an IgG3 in cell line YB2/0 confers a better capability to bond itself to CD16s through its Fc, while the same sequence expressed in the CHO line or expressed by the merged lymphocyte B (D29-P3X229) is bonded less better.

Furthermore, the expression of an IgG3 in line YB2/0 confers a capability to bond itself to the CD16 comparable to the capability of an IgG1 expressed in the same cell (R297 expressed in YB2/0) and also comparable with the capability of an IgG3 purified from an anti-D polyclonal antibody.

### Example 3

### Measurement by Competition of the Bonding of the Three Antibodies in Example 1 to Jurkat CD16 Cells by Flux Cytometry

Different dilutions of antibodies are incubated in the presence of Jurkat CD16 cells and the 3G8 anti-Cd16 antibody marked PE. The reactivity of antibodies to be evaluated with CD16 is inversely proportional to bonding of the 3G8 antibody marked PE that recognises the IgG bonding site to the CD16 Fc receptor. Thus, bonding to CD16 of unmarked antibodies to be evaluated will reduce bonding of the 3G8 antibody marked PE. Data are analysed and final results given as a percentage of bonding to CD16. FIG. 4 shows that the IgG1 and IgG3 antibodies produced in YB2/0 are comparably bonded to CD16 but more strongly than IgG3 antibodies produced in CHO or by lymphocyte B (D29-P3X229).

### Example 4

### Measurement of the Activation of Jurkat CD16 Cells (Experimental Follow Up of Example 2)

After evaluation of antibodies bonding to Jurkat CD16, the plates are then incubated for one night at 37° C. and then centrifuged. The quantity of IL2 released by Jurkat CD16 in culture media is evaluated using an ELISA technique.

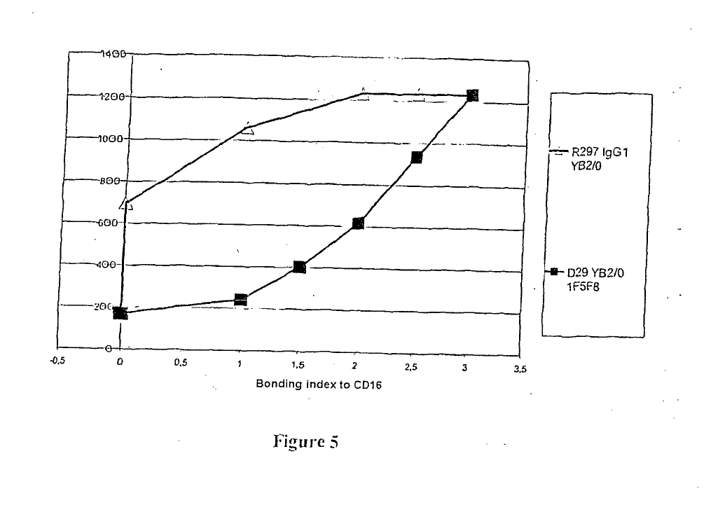

The results are given in quantity of IL2 as a function of the determined bonding to CD16 (example 2). FIG. 5 shows that interaction of IgG3s with Jurkat CD16 induces a much lower release of IL2 than in the presence of IgG1s. Thus, YB2/0 IgG1 induces a release of IL2 for the first bonding indexes, unlike YB2/0 IgG3; however, the response dose curve obtained with IgG3s is less than what is obtained with IgG1s. Only a strong interaction between YB2/0 IgG3s and CD16 (maximum bonding index of 3) induces a release of IL2 comparable to that obtained with IgG1s produced by YB2/0.

### Example 5

### Study of Cytotoxicity Induced by Anti-D Antibodies Against Positive Rh Red Cells in the Presence of PBMC or Purified Nk Cells

The PBMC cytolysis test quantifies the capability of antibodies to lyse Rhesus positive red cells in the presence of human mononuclear cells (PBMC) and polyvalent immunoglobulins (Tegelin).

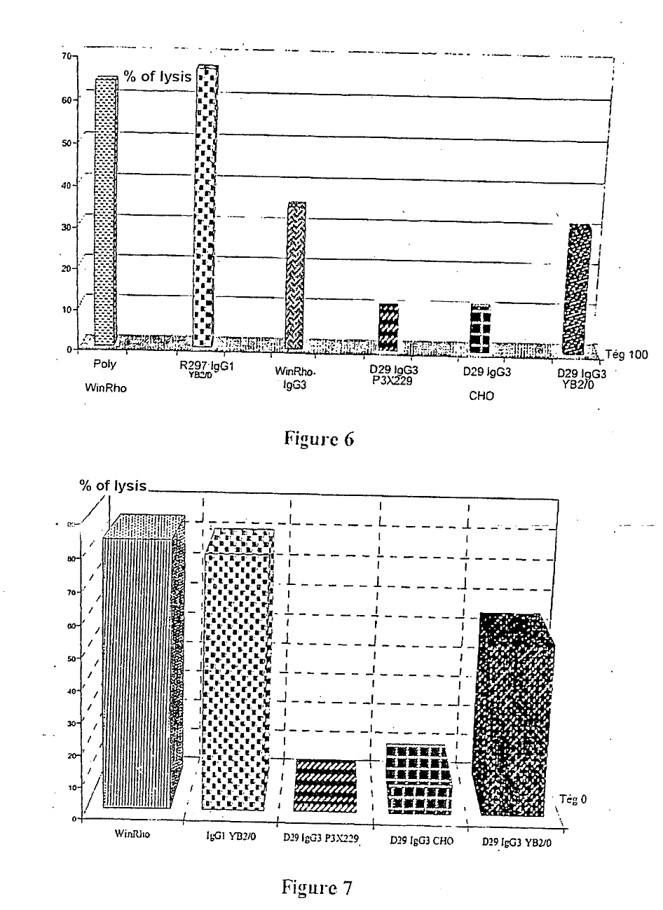

The results are given in FIG. 6.

The cytolytic activity of IgG3 expressed in CHO is comparable with that obtained with the antibody expressed by merged lymphocyte B D29-P3X229.

On the other hand, the expression of an IgG3 in YB2/0 potentialises its capacities to induce a lysis of red cells in the presence of mononucleated cells (PBL) by comparison with the same antibody produced in CHO or by heteromyeloma.

Compared with the activity of IgG3 produced in CHO, the increase in cytolytic activity of IgG3 expressed in YB20 is 2.8 times greater and is also comparable with that induced by the IgG3 polyclonal fraction of WinRho.

However, the cytolytic activity of IgG3s produced in YB2/0 is less than the cytolytic activity of IgG1s produced in YB2/0 and the anti-D polyclonal antibody (Poly-D WinRho).

In the presence of purified NK cells (FIG. 7), YB2/0 IgG3 induces a 5.5 times greater lysis of red cells (55%) than is obtained with the same antibody produced in CHO (10%). The antibody produced by heteromyeloma P3X229 gives the lowest value (4%).

Nevertheless, the cytolytic activity of IgG3s produced in YB2/0 is less than the cytolytic activity of IgG1s produced in YB2/0 and the WinRho polyclonal antibody.

### Example 6

### Measurement of the Release of IL-2 by Jurkat CD16 After Bonding of Antibodies to Red Cells in Suspension

Study of the inhibition of activation induced by YB2/0 IgG1s by addition of IgG3.

Release of IL2: Jurkat CD16 cells are incubated with Rhesus positive red cells, YB2/0 IgG1 or D29 IgG3 antibody expressed in different expression systems (YB2/0, CHO, B-P3X229). The IL2 release is measured in the floats after a night of incubation using the ELISA technique.

Results:

IgG3s expressed in YB2/0 and CHO do not induce any IL2 release, unlike YB2/0 IgG1s, for an identical concentration of antibodies (FIG. 8). We can deduce that unlike IgG1s expressed in YB2/0, bonding of an IgG3 expressed in YB2/0 on CD16 does not induce any release of IL2 in the presence of red cells in solution and Jurkat CD16 cells. Thus, example 4 in which the red cells were coated with microplates, showed that only a strong interaction with CD16 could induce a release of IL2. The use of more physiological conditions in this example confirms the very weak potential of YB2/0 IgG3s to induce a release of IL2 starting from Jurkat CD16, unlike YB2/0 IgG1s.

Inhibition study: Jurkat CD16 cells are incubated with Rhesus positive red cells and a YB2/0 IgG1 mixed with the different D29 IgG3s expressed in the different expression systems (YB2/0, CHO, B-P3X229). The release of IL2 is measured in the floats after a night of incubation using the ELISA technique.

Results:

IgG3s produced in CHO and P3X229 have no effect on the release of IL2 induced by YB2/0 IgG1. However, IgG3 produced in YB2/0 induces a reduction in the induction of IL2 (FIG. 8).

Therefore the expression of an IgG3 in YB2/0 induces a negative modulation of the activating activity of IgG1s.

### Example 7

### Induction of the Release of Cytokines by Anti-D Antibodies

Nk cells or monocytes are incubated with Rhesus positive red cells, the YB2/0 IgG1 or the D29 IgG3 antibody expressed in YB2/0 or CHO. The release of different cytokines (beta IL1, IL6, gamma IFN, alpha TNF) is measured in the floats after a night of incubation using the ELISA technique.

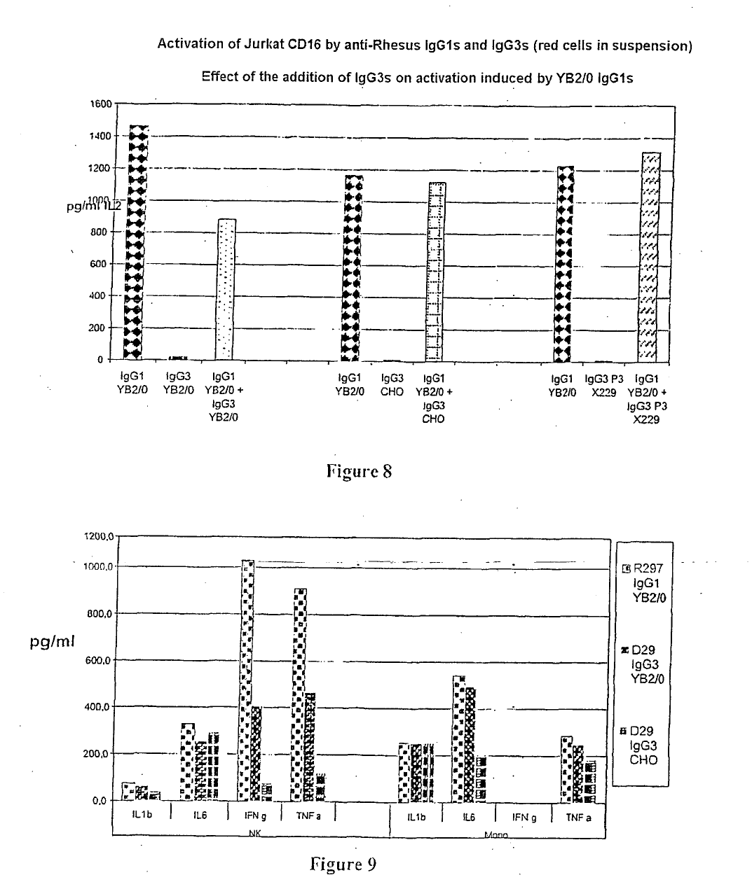

The results are shown in FIG. 9. In the presence of NK cells, the contents of beta IL1 and IL 6 are comparable for all antibodies. However, the contents of alpha TNF and gamma IFN produced by NK cells in the presence of IgG3 expressed by YB2/0 are less than the contents induced by IgG1 expressed in YB2/0 (FIG. 9). Nevertheless, this release is greater than the release observed for IgG3 produced by CHO.

In the presence of monocytes, the contents of beta IL1 and gamma IFN (zero) are identical for all antibodies. The contents of IL6 produced by monocytes are comparable for IgG1s and IgG3s produced in YB2/0 but are lower for IgG3 produced in CHO. A slight drop is observed for alpha TNF in the presence of IgG3 produced by CHO.

### Example 8

### Capability of Anti-D IgG3s to Induce a Phagocytosis of Rhesus Positive Red Cells by THP-1 Cells

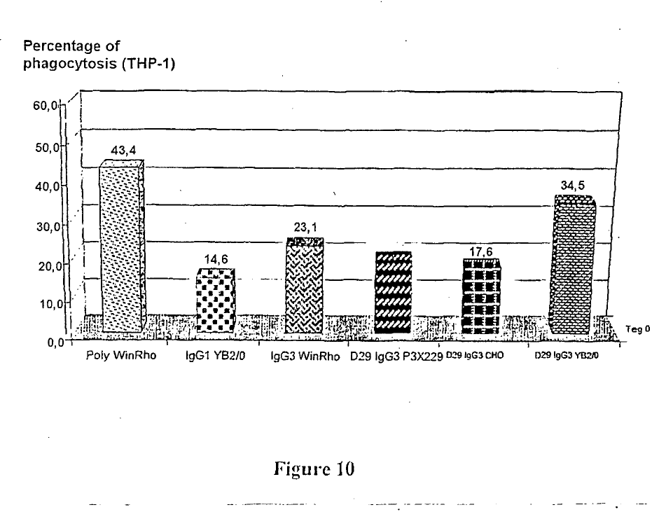

Phagocytosis test: THP-1 cells are incubated in the presence of Rhesus positive red cells and antibodies. The number of cells that have phagocyted at least one red cell is evaluated by counting on the microscope. Results are expressed as a percentage of cells that have phagocyted at least one red cell (see FIG. 10).

IgGs of the WinRho anti-D polyclonal antibody have the highest capability (43.4%) to induce phagocytosis of Rhesus positive red cells by cell THP-1. The YB2/0 IgG1 is only slightly active (14.6%). For IgG3s, YB2/0 IgG3 induces a phagocytosis of 34.5%, greater than purified polyclonal IgG3s (WinRho IgG3). The weakest phagocytosis activities are obtained with IgG3s produced by merged lymphocyte B (D29 P3X229) and IgG3 produced in CHO.

It can be concluded that the expression of an IgG3 in YB2/0 potentialises its capabilities of inducing phagocytosis, this property being particularly interesting for infectious diseases and Alzheimer's disease (McGeer P L, McGeer E. Immunotherapy for Alzheimer's diseases. Sci Aging Knowledge Environ. 2004 Jul. 7; 2004) and in comparison with an IgG3 expressed in CHO or released by a heteromyeloma.

Conclusion

The particular glycanic profiles of IgG3 produced in YB2/0, in other words short, non-sialyled forms with a fucose content of less than 35%, confers-innovative properties on it as demonstrated above:

a strong bonding to CD16 comparable to that of IgG1s,

potentialisation of phagocytosis,

an increase in the ADCC activity in the presence of PBMC or NK cells compared with IgG3s produced in CHO,

a capability to come into competition with IgG1s at their bonding to CD16 and thus negatively modulate the release of cytokines.

1. A method of treating cancer pathologies and infectious pathologies comprising administering chimeric, humanised or human class IgG3 monoclonal antibody produced in a cell line of rat myeloma, particularly YB2/0 (ATCC No. CRL 1662) or a derived or modified line of YB2/0 to a patient in need thereof.

2. The method of claim 1, wherein said patient exhibits a weak response to treatment with an IgG1 or an IgG3 expressed in CHO.

3. The method of claim 1, wherein said patient has a late diagnosis.

4. The method of claim 1, wherein said cancer pathologies are selected from the group consisting of neuroectodermal tumours, colorectal cancers, melanomas, breast cancer, leukaemia and HCL (Hairy Cell Leukaemia), lymphomas such as DLBCL (Primary Diffuse Large B-Cell Lymphomas), acute leukaemias, and osteosarcomas.

5. The method of claim 1, wherein said cancer pathologies are associated with viral or bacterial infections.

6. The method of claim 5, wherein viral or bacterial infections are selected from the group consisting of cancer of the prostate, leukaemias, and Kaposi's sarcoma.

7. The method of claim 1, wherein said infectious pathologies are selected from the group consisting of diphtheria, viral hemorrhagic fevers, typhoid fever, influenza, hepatitis B and C, respiratory infections due to RSV, infections due to HIV, legionnaires, disease, Leishmaniasis, leprosy, rabies, AIDS and tuberculosis.

8. The method of claim 1, wherein said antibody induces phagocytosis.

9. The method of claim 1, wherein said antibody is produced in a cell line of rat myeloma, particularly YB2/0 (ATCC No. CRL 1662) or a derived or modified line of YB2/0 is administered to said patient in combination with an IgG1.

10. The method of claim 1, wherein said antibody negatively modulates the release of cytokines induced by IgG1.

11. The method of claim 9, wherein said patient exhibits cancer pathologies consistent with a cytokine release syndrome.

12. The method of claim 11, wherein said patient suffers from hypothermia, acute renal necrosis or a diseases of the liver due to the cytokine release syndrome.

13. The method of claim 11, wherein the cytokine release syndrome has been induced by the administration of an anti-CD3 monoclonal antibody.

14. The method of claim 11, wherein said patient has been treated with said class IgG3 monoclonal antibody, which prevents the appearance of the cytokine release syndrome.

15. The method of claim 11, wherein said antibody prevents undesirable effects of alemtuzumab or OKT3 antibody.

16. A method for Process for modulating the release of cytokines induced by an IgG1, wherein IgG3s produced in a cell line of rat myeloma, particularly YB2/0, are added to a biological system containing said IgG1s.

17. The method of claim 16, wherein said IgG1s are produced in a cell line of rat myeloma, particularly YB2/0.

18. A pharmaceutical composition comprising IgG1s, IgG3s and at least one excipient.

19. The composition of claim 18, wherein at least one of said IgG1s or IgG3s is produced in a rat myeloma cell line.

20. The method of claim 10, wherein said antibodies negatively modulate the release of gamma IFN, alpha TNF and/or IL6 cytokines induced by IgG1.

21. The composition of claim 19, wherein said at least one of said IgG1s or IgG3s is produced in the rat myeloma cell line YB2/O.
